(19) United States
(12) Patent Application Publication
(10) Pub. No.: US 2020/0118685 A1
(43) Pub. Date: Apr. 16, 2020

(54) METHOD AND APPARATUS FOR PROVIDING USER HEALTH STATUS

(71) Applicant: UNIVERSITY-INDUSTRY COOPERATION GROUP OF KYUNG HEE UNIVERSITY, Gyeonggi-do (KR)

(72) Inventors: Sung Young LEE, Gyeonggi-do (KR); Hafiz Syed Muhammad BILAL, Gyeonggi-do (KR)

(21) Appl. No.: 16/422,788

(22) Filed: May 24, 2019

Related U.S. Application Data
(60) Provisional application No. 62/770,581, filed on Nov. 21, 2018.

(30) Foreign Application Priority Data
May 30, 2018 (KR) 10-2018-0061516
Mar. 15, 2019 (KR) 10-2019-0029815

(51) Int. Cl. G16H 50/30; A61B 5/0488; A61B 5/024; A61B 5/0402; G16H 10/60; A61B 5/0476

(52) U.S. Cl. CPC G16H 50/30; A61B 5/0488; A61B 5/0476; A61B 5/0402; G16H 10/60; A61B 5/024

(57) ABSTRACT

Disclosed is a user health status providing method in which a server provides a user health status, the method including: receiving user behavior data from a user device; extracting health data from the behavior data, and matching the health data to one or more health groups; generating first health indices associated with the health groups using healthcare models specified in advance for the health groups and the health data; generating a second health index by integrating the one or more first health indices, each of which is generated for one of the health groups; and determining the user health status on the basis of the second health index.

### BACKGROUND OF THE INVENTION

### 1. Field of the Invention

The present disclosure relates to a user health status providing method and apparatus, and more particularly, to a method and apparatus for monitoring a user health status using user health data received from a user device.

### 2. Description of the Prior Art

In the fields of technical development, education, entertainment, finance, business, and medical treatment, data may be personalized via self-quantification and digitization. Data digitization may quantify user data so as to track all patterns that can support identifying the health status and behavior status of a user. The development of data digitization technology has enabled behavior monitoring that analyzes unhealthy patterns in a user's daily life routine.

Existing programs quantize data directly related to a user's life expectancy, such as diet, physical activity, smoking, drinking, or the like, analyze user data, and provide an analysis result to the interested parties. For the purpose of health data management, the existing programs may monitor and store important statistics. In this instance, a technique of removing noise from collected data and filtering out only information related to health among user behavior data is narrowly applied, and the existing programs are dependent upon individual experts in order to provide a customized health status monitoring service suited to a user, which are drawbacks.

### SUMMARY OF THE INVENTION

The present disclosure has been made in order to solve the above-mentioned problems in the prior art and an aspect of the present disclosure is to provide a method and an apparatus for analyzing user behavior data and determining a health status.

Another aspect of the present disclosure is to provide a method and an apparatus for classifying user behavior data as one or more health groups, and analyzing the behavior data using a model different for each health group.

In accordance with an aspect of the present disclosure, a user health status providing method in which a server provides a user health status includes: receiving user behavior data from a user device; extracting health data from the behavior data, and matching the health data to one or more health groups; generating first health indices associated with the health groups using healthcare models specified in advance for the health groups and the health data; generating a second health index by integrating the one or more first health indices, each of which is generated for one of the health groups; and determining the user health status on the basis of the second health index.

The operation of receiving the user behavior data includes: receiving sensor values of a plurality of sensors included in the user device; and receiving, from the user device, user health information obtained via a user input.

The operation of generating the second health index includes: extracting a weight for each health group on the basis of a degree that the health group affects the user health status; and generating the second health index by multiplying the one or more first health indices by the weights, and adding result values.

The method further includes: providing a healthcare report including the health status and the first health index generated for each health group.

The health group includes one or more of a physical activity status, a calorie intake status, a drinking status, a smoking status, and a stress status.

The healthcare model for each health group generates the first health index by using a user activity time when the health group is the physical activity status, by using a user intake of nutrients when the health group is the calorie intake status, by using a user intake of alcohol when the health group is the drinking status, by using a user intake of smoking when the health group is the smoking status, and by using one or more sensor values among a user heart rate, electrocardiogram, electroencephalogram, and electromyogram when the health group is the stress status.

The operation of determining the user health status includes: determining the user health status by comparing the second health index with a first threshold value and a second threshold value, which are set in advance.

The method further includes: determining the user health status to be unhealthy when the second health index is less than the first threshold value; determining the user health status to be moderate when the second health index is greater than or equal to the first threshold value and less than the second threshold value set in advance; and determining the user health status to be healthy when the second health index is greater than or equal to the second threshold value.

In accordance with an aspect of the present disclosure, a user health status providing apparatus for monitoring a user health status includes: a data reception unit configured to receive user behavior data from a user device; a data analysis unit configured to extract health data from the behavior data, and to match the health data to one or more health groups; and a health status evaluation unit configured to generate first health indices associated with the health groups using healthcare models specified in advance for the health groups and the health data, to generate a second health index by integrating the one or more first health indices, each of which is generated for one of the health groups, and to determine the user health status on the basis of the second health index.

The data reception unit is configured to receive sensor values of a plurality of sensors included in the user device, and to receive, from the user device, user health information obtained via a user input.

The health status evaluation unit includes a health index generation unit which is configured to extract a weight for each health group on the basis of a degree that the health group affects the user health status, and to generate the second health index by multiplying the one or more first health indices by the weights and adding result values.

The health status evaluation unit is configured to generate a healthcare report including the health status and the first health index generated for each health group.

The health group includes one or more of a physical activity status, a calorie intake status, a drinking status, a smoking status, and a stress status.

The healthcare model for each health group generates the first health index by using a user activity time when the health group is the physical activity status, by using a user intake of nutrients when the health group is the calorie intake status, by using a user intake of alcohol when the health group is the drinking status, by using a user intake of smoking when the health group is the smoking status, and by using one or more sensor values among a user heart rate, electrocardiogram, electroencephalogram, and electromyogram when the health group is the stress status.

The health status evaluation unit is configured to determine the user health status by comparing the second health index with a first threshold value and a second threshold value, which are set in advance.

The health status evaluation unit performs: determining the user health status to be unhealthy when the second health index is less than the first threshold value; determining the user health status to be moderate when the second health index is greater than or equal to the first threshold value and less than the second threshold value set in advance; and determining the user health status to be healthy when the second health index is greater than or equal to the second threshold value.

According to the present disclosure, user behavior data is analyzed so as to determine a health status.

According to the present disclosure, user behavior data is classified as one or more health groups and the behavior data is analyzed using a model different for each health group.

### BRIEF DESCRIPTION OF THE DRAWINGS

The above and other aspects, features and advantages of the present disclosure will be more apparent from the following detailed description taken in conjunction with the accompanying drawings, in which:

FIG. 1 is a diagram illustrating the configuration of a user health status providing device according to an embodiment of the present disclosure; and

FIG. 2 is a diagram illustrating a user health status providing method according to an embodiment of the present disclosure.

### DETAILED DESCRIPTION OF THE EXEMPLARY EMBODIMENTS

The above-described aspects, features, and advantages will be described with reference to enclosed drawings. Accordingly, those skilled in the art may easily implement the technical idea of the present disclosure. When detailed descriptions related to a well-known related art are determined to make the subject of the present disclosure ambiguous, the detailed descriptions will be omitted herein.

The same reference numerals in the drawings denote the same or like elements. All combinations described in the specification and the scope of the claims may be combined based on a random method. Singular forms are intended to include the plural forms as well, unless the context clearly indicates otherwise.

Terms used in the present specification are merely used for the purpose of describing specific exemplary embodiments and are not intended to limit the present disclosure. In the present specification, the expressions provided in the singular form may be intended to include the meaning of the plural form, unless otherwise specified in the corresponding sentence. The term “and/or” may include all combinations of enumerated items and one of the items. The term “comprise”, “comprising”, “equipped with”, “have”, “having”, or the like may include have the meaning of inclusion. Accordingly, the terms may specify the specified feature, integer, step, operation, element, and/or component, and may not exclude the existence or addition of one or more other features, integers, steps, operations, elements, components, and/or groups thereof. Steps, processes, and operations of the method described in the present specification should not be construed to be performed in a specific order which has been discussed or illustrated in the present disclosure, unless the order of performance is decided. It should be understood that there may be additionally or alternatively steps.

Also, each element may be implemented as a hardware processor. Respective elements may be implemented as a single hardware processor via integration. Alternatively, respective elements may be combined and may be implemented as a plurality of hardware processors.

Hereinafter, exemplary embodiments according to the present disclosure will be described in detail with reference to enclosed drawings.

Nowadays the focus of the software industry is to develop technology that analyzes and visualizes the tendencies of user behavior during his/her daily activities. However, even through user behavior is analyzed, a user may not be easy to recognize the meaning implied in the analyzed result since the user may not have expert knowledge associated with health. Therefore, the present disclosure is to provide customized medical treatment and well-being service suited to a user by performing quantitative analysis on user behavior. Particularly, the present disclosure is to analyze user behavior data (life log), to extract user habits, and to provide the same to a user, so that the user may recognize user behavior and habits.

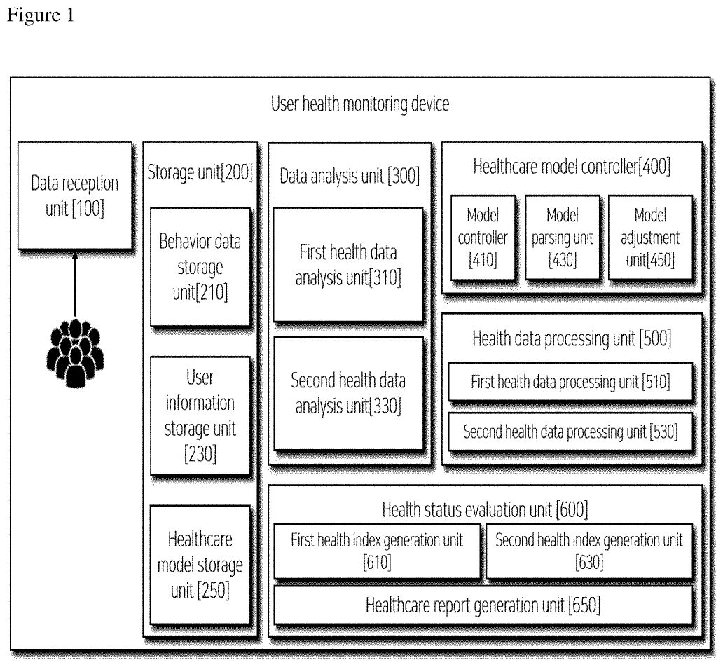

FIG. 1 is a diagram illustrating the configuration of a user health status providing device according to an embodiment of the present disclosure.

Referring to FIG. 1, the user health status providing device may include a data reception unit 100, a storage unit 200, a data analysis unit 300, a healthcare model controller 400, a health data processor 500, and a health status evaluation unit 600. The user health status providing device may be implemented as a server. Accordingly, hereinafter, the user health status providing device is referred to as a server.

The data reception unit 100 may receive user behavior data from a user device. The user behavior data may be a value that a sensor included in the user device measures or may be health information that is input via the user device. The user device may include a plurality of sensors, for example, a pedometer, a gyro sensor, an acceleration sensor, a cardiotachometer, an electrocardiogram measurement instrument, a brain wave monitor, an electromyogram measurement instrument, a weight sensor, a temperature sensor, a humidity sensor, an illumination sensor, and the like. The user device may be a sensor hub existing in a smart device (e.g., a smart phone, a tablet PC, or the like) or a wearable device. The user device may receive a sensor value associated with user's physical information and behavior information and may transmit the same to the server.

In addition, the data reception unit 100 may receive user unique information, such as gender, age, physical information and the like from the user device.

The user behavior data and/or user unique information received via the data reception unit 100 may be stored in the storage unit 200.

The storage unit 200 may include a behavior data storage unit 210, a user information storage unit 230, and a healthcare model storage unit 250. The behavior data storage unit 210 may store behavior data received via the data reception unit 100. The user information storage unit 230 may store user unique information received via the data reception unit 100. In addition, the healthcare model storage unit 250 may store at least one healthcare model used for analyzing user behavior data.

The data analysis unit 300 may analyze behavior data stored in the behavior data storage unit 210, and may indicate user behavior using a quantitative value. The data analysis unit 300 may include a first health data analysis unit 310 and a second health data analysis unit 330.

The data analysis unit 300 may extract health data indicating a health status from behavior data, and may classify the health data as first health data and second health data. The first health data analysis unit 310 may identify the first health data in extracted health data. The first health data may indicate health data measured by a sensor included in the user device. In addition, the second health data analysis unit 330 may identify the second health data in extracted health data. The second health data analysis unit 330 may identify health data remaining after excluding the first health data, as the second health data. The second health data may indicate health data that a user inputs via the user device. In this instance, the second health data may include a calorie intake, an intake of drinking, and an intake of smoking.

In addition, the data analysis unit 300 may match health data to one or more health groups. The health groups may include a physical activity status, a calorie intake status, a drinking status, a smoking status, and a stress status. The health groups may not be limited to the above-mentioned five categories, and more categories may be added thereto or deleted therefrom.

The first health data analysis unit 310 may classify the first health data as the physical activity status and stress status health groups, and the second health data analysis unit 330 may classify the second health data as the calorie intake status, drinking status, and smoking status health groups.

The healthcare model controller 400 may parse a healthcare model to be applied to health data classified for each health group. In this instance, the healthcare model may be configured to be different for each health group. The healthcare model controller 400 may include a model controller 410, a model parsing unit 430, and a model adjustment unit 450.

The model controller 410 may access the healthcare model storage unit 250 and retrieve a healthcare model so as to quantitatively analyze health data. The model parsing unit 430 may parse a healthcare model specified for a health group which health data belongs to.

The model adjustment unit 450 provides an interface that an expert may access so as to add, modify, or delete a healthcare model using knowledge that the expert possesses. The model adjustment unit 450 may store a healthcare model that an expert adjusts via an interface, in the healthcare model storage unit 250.

The health data processing unit 500 may extract one or more parameters from health data in order to apply a healthcare model to health data. A first health data processing unit 510 may extract a parameter from first health data, and a second health data processing unit 530 may extract a parameter from second health data.

The first health data processing unit 510 may extract parameters of health data associated with the physical activity status and health data associated with the stress status, which correspond to the first health data. The first health data processing unit 510 may extract a user activity time when a MET is greater than or equal to 3, from the health data associated with the physical activity status, and may extract measurement values, obtained by a cardiotachometer, an electrocardiogram measurement instrument, a brain wave monitor, an electromyogram measurement instrument, from the health data associated with the stress status.

The second health data processing unit 530 may extract parameters of health data associated with the calorie intake status, health data associated with the drinking status, and health data associated with the smoking status, which correspond to the second health data. The second health data processing unit 530 may extract an intake of fruits, vegetables, dairy products, fat, salt, sugar, grains, and meat, from health data associated with the calorie intake, and may retrieve diet rule information and food intake information of a user from the user information storage unit 230. The diet rule information may indicate a time when a user eats a meal. The food intake information may indicate nutrients that a user takes in. The food intake information may be used for determining whether a user ingests a sufficient amount of carbohydrate, protein, fat, vitamin, and mineral, and may be classified as five groups, such as grains, poultry, dairy products, fruits, and vegetables.

The second health data processing unit 530 may extract user's intake of drinking from the health data associated with the drinking status, and may extract user's intake of smoking from the health data associated with the smoking status. In this instance, the intake of drinking may indicate the amount of alcohol that a user drinks, and the intake of smoking may indicate the number of smoking products that a user consumes.

The health status evaluation unit 600 may quantitatively analyze health data by applying, to the health data, a healthcare model parsed by the healthcare model controller 400, and may determine the health status of a user. More particularly, the health data evaluation unit 600 may include a first health index generation unit 610 that quantitatively analyzes health data, a second health index generation unit 630 that generates integrated second health data associated with user health data by integrating first health indices, each of which is generated for one of the health groups, and a healthcare report generation unit 650 that analyzes a second health index so as to determine the health status of a user, and generates a healthcare report that visually shows the health state of the user. The first health index generation unit 610 may generate a first health index using a parameter for each health data according to a health group to which the health data belongs. The first health index generation unit 610 may quantitatively analyze health data by applying a healthcare model specified for a health group which the health data belongs to, and may generate a first health index for each health group. Particularly, the first health index generation unit 610 may generate a first health index associated with the physical activity status using a healthcare model specified for the health group of the physical activity status, in association with health data matched to the health group of the physical activity status. In this instance, the healthcare model specified for the health group of the physical activity status may generate the first health index associated with the physical activity using a time when a MET is greater than or equal to 3, as a parameter, as shown in Equation 1. The metabolic Equivalent (MET) is the standardization that expresses the intensity of various physical activities. A MET less than 3 indicates a low intensity physical activity. A MET greater than or equal to 3 and less than 6 indicates a medium intensity physical activity. A MET greater than or equal to 6 indicates a high intensity physical activity. The present disclosure may set a criterion for determining the physical activity status of a user to MET=3, and thus, it is determined that the user is in the physical activity status when the user performs a physical activity of a medium intensity. In this instance, the criterion associated with the MET may be changed depending on a setting.

B_{phyAct}=Σ(time_{phyAct}|MET_{phyAct}≥3)/Week [Equation 1]

When health data is matched to the health group of the stress status, the first health index generation unit 610 may generate a first health index associated with the stress status using a healthcare model specified for the health group of the stress status. The healthcare model specified for the health group of the stress status may generate the first health index associated with the stress status which indicates the degree of stress that a user's body receives, using measurement values obtained by a cardiotachometer, an electrocardiogram measurement instrument, a brain wave monitor, and an electromyogram measurement instrument, as parameters.

When health data is matched to the health group of the calorie intake status, the first health index generation unit 610 may generate a first health index associated with the calorie intake status using a healthcare model specified for the health group of the calorie intake status. In this instance, the healthcare model specified for the health group of the calorie intake status may generate the first health index associated with the calorie intake status using an intake of fruits, an intake of vegetables, an intake of dairy products, an intake of fat, an intake of salt, an intake of sugar, an intake of grains, an intake of meat, eating regularity, and balanced diet. The healthcare model specified for the health group of the calorie intake may be as shown in Equation 2.

$${B}_{Diet} = ( {DietryHabits}_{SCORE} + {DietryNutrient}_{SCORE} ) , \\ {DietryNutrient}_{SCORE} = \frac{\begin{matrix}\{ Fruit + Vegetable + Dairy + Fat + \\ Salt + Sugar + Wholegrain + Poultry \}\end{matrix}}{Week} \qquad [ Equation 2 ]$$

When health data is matched to the health group of the drinking status, the first health index generation unit 610 may generate a first health index associated with the drinking status using a healthcare model specified for the health group of the drinking status. In this instance, the healthcare model specified for the health group of the drinking status may use an intake of drinking, which is received from a user, as shown in Equation 3.

B_{Alcohol}=no. of Drinks/Week [Equation 3]

When health data is matched to the health group of the smoking status, the first health index generation unit 610 may generate a first health index associated with the smoking status using a healthcare model specified for the health group of the smoking status. In this instance, the healthcare model specified for the health group of the smoking status may use an intake of smoking, which is received from a user, as shown in Equation 4.

B_{Smoking}=no. of Packs(cigarette)/Week [Equation 4]

The second health index generation unit 630 may generate a second health index by integrating a plurality of first health indices generated by the first health index generation unit 610. The second health index generation unit 630 may generate the second health index using a weight assigned for each health group corresponding to health data. A weight may be set to be different for each user. In this instance, user's age, physical condition, recent physical examination records, and previous healthcare report may be used as reference data. The second health index generation unit 630 may generate the second health index (health behavior index (HBI)) via Equation 5.

HBI=Σᵢ₌₁ⁿ{Bᵢ*Wt_{B}_{ᵢ}} [Equation 5]

The healthcare report generation unit 650 may generate a healthcare report using a first health index and a second health index. The healthcare report may include the second health index, and may provide user's overall health status. The healthcare report generation unit 650 may determine that the health status of the user is unhealthy when the value of the second health index is less than a first threshold value, may determine that the health status of the user is moderate when the value of the second health index is greater than or equal to the first threshold value and less than a second threshold value, and may determine that the health status of the user is healthy when the value of the second health index is greater than or equal to the second threshold value. For example, the healthcare report generation unit 650 may determine that the health status of the user is unhealthy when the value of the second health index is less than 12, may determine that the health status of the user is moderate when the value of the second health index is greater than or equal to 12 and less than 29, and may determine that the health status of the user is healthy when the value of the second health index is greater than or equal to 29 and less than 40. A reference threshold value of the second health index, used for determining the health status of a user, may be changed depending on a setting.

$${Status}_{HBI} = \{ \begin{matrix}unhealthy , & if < HBI < 12 \\ moderate , & if 12 < HBI < 29 \\ healthy , & if 29 < HBI < 40\end{matrix} \qquad [ Equation 6 ]$$

The healthcare report generation unit 650 may provide a first health index for each health group, and may provide the detailed health status of the user.

According to another embodiment of the present disclosure, the present disclosure may not be limited to provision of a healthcare report, and may further include transmission of an expert advice to a user. For example, when a first health index for each health group of a user exceeds a predetermined reference, the server may inquire of a related medical institution, and an expert belonging to the medical institution may transmit advice suitable for the user to the server. The server may receive a plurality of pieces of expert advice and integrate the same so as to provide the same to the user. In this instance, the server may provide expert advice in order of priority by taking into consideration of user's position, age, physical condition, and the like.

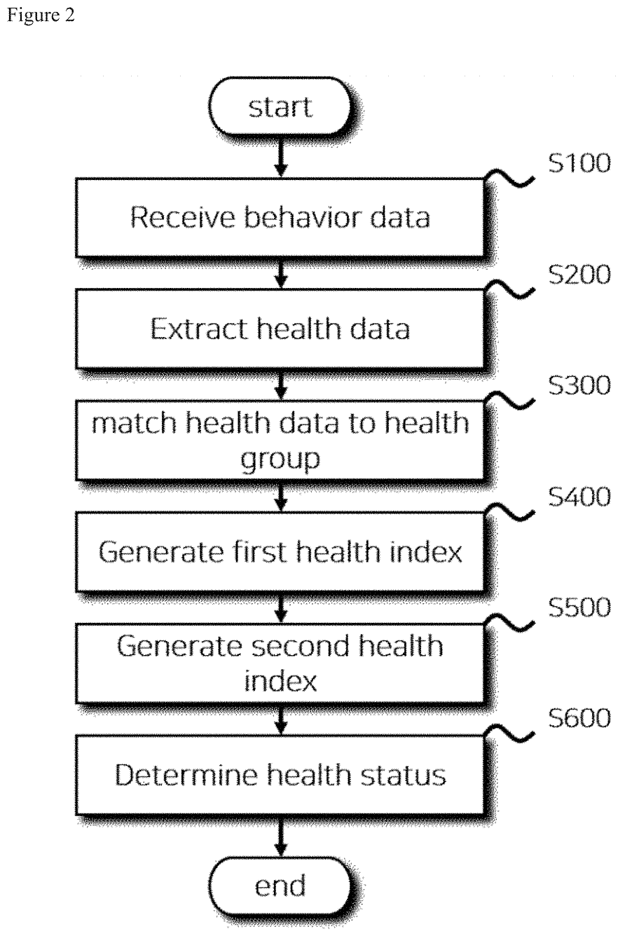

Hereinafter, a user health status providing method according to an embodiment of the present disclosure will be described with reference to FIG. 2. In the descriptions of the user health status providing method, detailed embodiments that overlap with the descriptions of the user health status providing system will be omitted.

FIG. 2 is a diagram illustrating a user health status providing method according to an embodiment of the present disclosure. Referring to FIG. 2, a server may receive user behavior data from a user device in operation S100. The server may receive sensor values of a plurality of sensors included in the user device, or may receive user health information obtained via a user input, from the user device.

The server may extract health data from the received behavior data in operation S200, and may match the health data to one or more health groups in operation S300. The health group may be classified in advance, and may include one of a physical activity status, a calorie intake status, a drinking status, a smoking status, and a stress status.

The server may generate a first health index for each health group by applying a healthcare model specified for each health group to health data matched to one or more health groups in operation S400. In order to apply a healthcare model to health data, the server may extract one or more parameters from the health data, and may apply the same to the healthcare model. The parameters extracted from the health data may be different for each health group.

The server may generate a second health index by integrating one or more first health indices, each of which is generated for one of the health groups, in operation S500. In order to generate the second health index, the server may extract a weight for each health group. The weight may be set to be different depending on a degree that a health group affects a user health status.

The server may determine the user health status by comparing the second health index with a first threshold value and a second threshold value, which are set in advance, in operation S600, and may generate a healthcare report including the user health status. The healthcare report may further include the one or more first health indices associated with health groups, so that the user may accurately recognize the health status.

Embodiments of the present disclosure provided in the present specification and the accompanying drawings are just predetermined examples for easily describing the technical contents of the present disclosure and helping understanding of present disclosure, but the present disclosure is not limited thereto. It is apparent to those skilled in the technical field of the present disclosure that other modifications based on the technical idea of the present disclosure are possible.

What is claimed is:

1. A user health status providing method in which a server provides a user health status, the method comprising:
Receiving user behavior data from a user device;
Extracting health data from the behavior data, and matching the health data to one or more health groups;
Generating first health indices associated with the health groups using healthcare models specified in advance for the health groups and the health data;
Generating a second health index by integrating the one or more first health indices, each of which is generated for one of the health groups; and
Determining the user health status on the basis of the second health index.

2. The method of claim 1, wherein the receiving the user behavior data comprises:
Receiving sensor values of a plurality of sensors included in the user device; and
Receiving, from the user device, user health information obtained via a user input.

3. The method of claim 1, wherein the generating the second health index comprises:
Extracting a weight for each health group on the basis of a degree that the health group affects the user health status; and
Generating the second health index by multiplying the one or more first health indices by the weights, and adding result values.

4. The method of claim 1, further comprising:
Providing a healthcare report including the health status and the first health index generated for each health group.

5. The method of claim 1, wherein the health group includes one or more of a physical activity status, a calorie intake status, a drinking status, a smoking status, and a stress status.

6. The method of claim 5, wherein the healthcare model for each health group generates the first health index by using a user activity time when the health group is the physical activity status, by using a user intake of nutrients when the health group is the calorie intake status, by using a user intake of alcohol when the health group is the drinking status, by using a user intake of smoking when the health group is the smoking status, and by using one or more sensor values among a user heart rate, electrocardiogram, electroencephalogram, and electromyogram when the health group is the stress status.

7. The method of claim 1, wherein the determining the user health status comprises:
Determining the user health status by comparing the second health index with a first threshold value and a second threshold value, which are set in advance.

8. The method of claim 7, further comprising:
Determining the user health status to be unhealthy when the second health index is less than the first threshold value;
Determining the user health status to be moderate when the second health index is greater than or equal to the first threshold value and less than the second threshold value set in advance; and
Determining the user health status to be healthy when the second health index is greater than or equal to the second threshold value.

9. A user health status providing apparatus for monitoring a user health status, the apparatus comprising:
A data reception unit configured to receive user behavior data from a user device;
A data analysis unit configured to extract health data from the behavior data, and to match the health data to one or more health groups; and
A health status evaluation unit configured to generate first health indices associated with the health groups using healthcare models specified in advance for the health groups and the health data, to generate a second health index by integrating the one or more first health indices, each of which is generated for one of the health groups, and to determine the user health status on the basis of the second health index.

10. The apparatus of claim 9, wherein the data reception unit is configured to receive sensor values of a plurality of sensors included in the user device, and to receive, from the user device, user health information obtained via a user input.

11. The apparatus of claim 9, wherein the health status evaluation unit includes a health index generation unit which is configured to extract a weight for each health group on the basis of a degree that the health group affects the user health status, and to generate the second health index by multiplying the one or more first health indices by the weights and adding result values.

12. The apparatus of claim 9, wherein the health status evaluation unit is configured to generate a healthcare report including the health status and the first health index generated for each health group.

13. The apparatus of claim 9, wherein the health group includes one or more of a physical activity status, a calorie intake status, a drinking status, a smoking status, and a stress status.

14. The apparatus of claim 13, wherein the healthcare model for each health group generates the first health index by using a user activity time when the health group is the physical activity status, by using a user intake of nutrients when the health group is the calorie intake status, by using a user intake of alcohol when the health group is the drinking status, by using a user intake of smoking when the health group is the smoking status, and by using one or more sensor values among a user heart rate, electrocardiogram, electroencephalogram, and electromyogram when the health group is the stress status.

15. The apparatus of claim 9, wherein the health status evaluation unit is configured to determine the user health status by comparing the second health index with a first threshold value and a second threshold value, which are set in advance.

16. The apparatus of claim 15, wherein the health status evaluation unit performs:
Determining the user health status to be unhealthy when the second health index is less than the first threshold value;
Determining the user health status to be moderate when the second health index is greater than or equal to the first threshold value and less than the second threshold value set in advance; and
Determining the user health status to be healthy when the second health index is greater than or equal to the second threshold value.
